(19) United States
(12) Patent Application Publication
(10) Pub. No.: US 2012/0265312 A1
(43) Pub. Date: Oct. 18, 2012

(54) System and Method for Manufacture of a Cranial Repair Implant and a Cranial Repair Implant Produced Thereby

(76) Inventors: Shawn Burke, Jacksonville, FL (US); Michael Teague, Jacksonville, FL (US); Pat Lemoyne, Jacksonville, FL (US)

(21) Appl. No.: 13/433,918

(22) Filed: Mar. 29, 2012

Related U.S. Application Data
(60) Provisional application No. 61/516,338, filed on Apr. 1, 2011.

(51) Int. Cl. A61F 2/28

(52) U.S. Cl. 623/17.19

(57) ABSTRACT

A custom cranial repair implant is prepared by conforming a malleable sheet material, such as for example a thin metal foil, a polymer sheet material or a laminate of various materials, a formable cement or putty-like material, or a spray or liquid settable material, to the edges and depth of a cranial defect in a manner that creates a mold matching the cranial defect. The mold is removed from the cranial defect and a settable putty or cement material that is biocompatible is then poured into the mold and allowed to harden. Once sufficiently cured, the implant is then removed from the mold and further shaped if necessary by cutting, abrading or the like. The implant is then affixed within the cranial defect in known manner, such as by bonding, gluing, mechanical fasteners, etc.

This application claims the benefit of U.S. Provisional Patent Application Ser. No. 61/516,338, filed Apr. 1, 2011, the disclosure of which is incorporated herein by reference.

### BACKGROUND OF THE INVENTION

This invention relates generally to the field of artificial implants for bone repair, and more particularly relates to implants that replace damaged or missing portions of bones, and even more particularly relates to implants for use in repairing the cranium. The invention relates generally to the field of such implants themselves, to the field of methodologies for forming such implants, in particular to any methodologies for forming the implant in the operating theater, and to systems comprising apparatuses and compositions for forming such implants.

In many situations it is medically necessary to replace a relatively large portion of the cranium that has either been removed by a surgeon for access to the brain or to address damaged portions of the cranium resulting from disease, accident or physical attack. Whereas a surgeon may be able to remove a portion of the skull in a controlled shape or configuration, such as for example a circle, triangle or the like, in certain instances the missing cranial portion will be irregularly shaped and a custom implant needs to be provided. In the first instance, a pre-formed replacement implant may be available that approximates the needed the shape, but in the second instance obtaining a properly configured implant is difficult.

In one known method addressing the problem of irregularly shaped implants, a CT, MRI or similar type scan is performed on the patient, the scan information is sent offsite and a properly configured custom implant is produced and shipped back for implantation. An example of this method is shown in U.S. Pat. No. 7,050,877 to Iseki et al. While this method produces a replacement implant that conforms well with the missing cranial portion, the production delay means that the implant is not available during a first procedure and a second procedure must subsequently be performed to affix the implant. Another method currently in use is to provide an assortment of pre-formed implants in varying shapes and dimensions. The surgeon then chooses the best fit to implant. The problem with this system is that the implants are all variations of common shapes and none of the assortment is likely to provide a good match for an irregular cranial defect. Still another known method is shown in U.S. Pat. No. 5,503,164 to Friedman, where a malleable scaffold is shaped and mechanically fastened to the cranial defect and the scaffold is then filled with a bone cement or putty to fill in the defect. The problem with this system is that the scaffolding and mechanical fasteners remain attached to the cranium.

It is an object of this invention to provide a system, methodology and implant that addresses the problems discussed above, wherein the invention provides for a custom implant that conforms well to any cranial defect, whether irregular or regular in configuration, and wherein the implant is produced at the time and location where the implantation is to occur.

### SUMMARY OF THE INVENTION

A custom cranial repair implant is prepared by conforming a malleable sheet material, such as for example a thin metal foil, a polymer sheet material or a laminate of various materials, a formable cement or putty-like material, or a spray or liquid settable material, to the edges and depth of a cranial defect in a manner that creates a mold matching the cranial defect in both peripheral configuration and depth. The material utilized to create the mold is such that either by inherent rigidity or subsequent curing, the mold retains it shape once removed from the cranial defect. A settable putty or cement material that is biocompatible is then poured into the mold and allowed to harden. Once sufficiently cured, the implant is removed from the mold and further shaped if necessary by cutting, abrading or the like. The implant is then affixed within the cranial defect in known manner, such as by bonding, gluing, mechanical fasteners, etc.

### BRIEF DESCRIPTION OF THE DRAWINGS

FIG. 1 view of a cranium showing a defect requiring an implant.

FIG. 2 is a front view of the irregularly shaped cranial defect.

FIG. 3 is a front view showing the malleable mold forming material positioned within and conforming to the configuration of the cranial defect.

FIG. 4 is a cross-sectional view taken along line IV-IV of FIG. 3.

FIG. 5 is a cross-sectional view showing the curable implant forming material positioned within the mold, now removed from the cranium.

FIG. 6 is a cross-sectional view showing the cured implant removed from the mold.

FIG. 7 is a front view showing the implant positioned within the cranial defect and ready for affixation.

### DETAILED DESCRIPTION OF THE INVENTION

With reference to the drawings, the system, method and article of manufacture embodiments for the invention will now be described in detail. In general, the method of repairing a cranial defect comprises the steps of providing a mold forming member; manipulating the mold forming member within a cranial defect in a cranium such that the mold folding member conforms to the shape of the cranial defect and forms a negative mold; removing the mold from the cranial defect; providing an implant forming material; filling the mold with the implant forming material; allowing the implant forming material to cure to form an implant; removing the implant from the mold; positioning the implant in the cranial defect; and affixing the implant to the cranium.

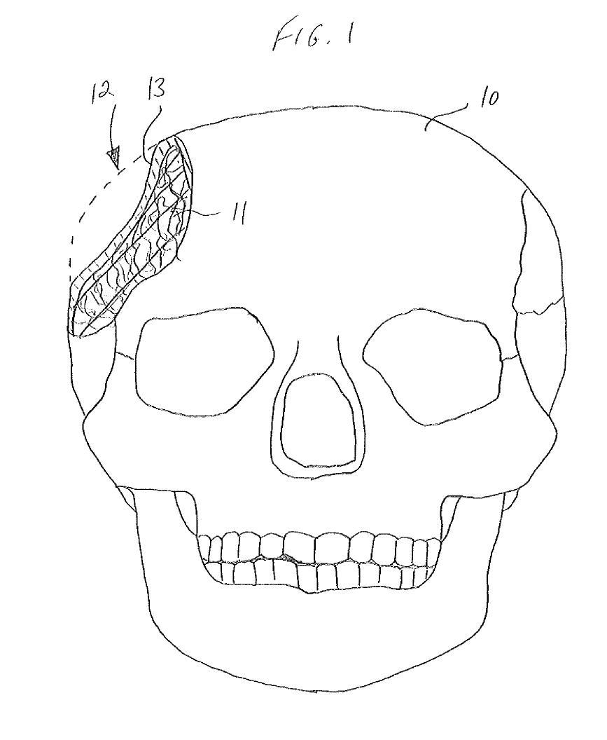

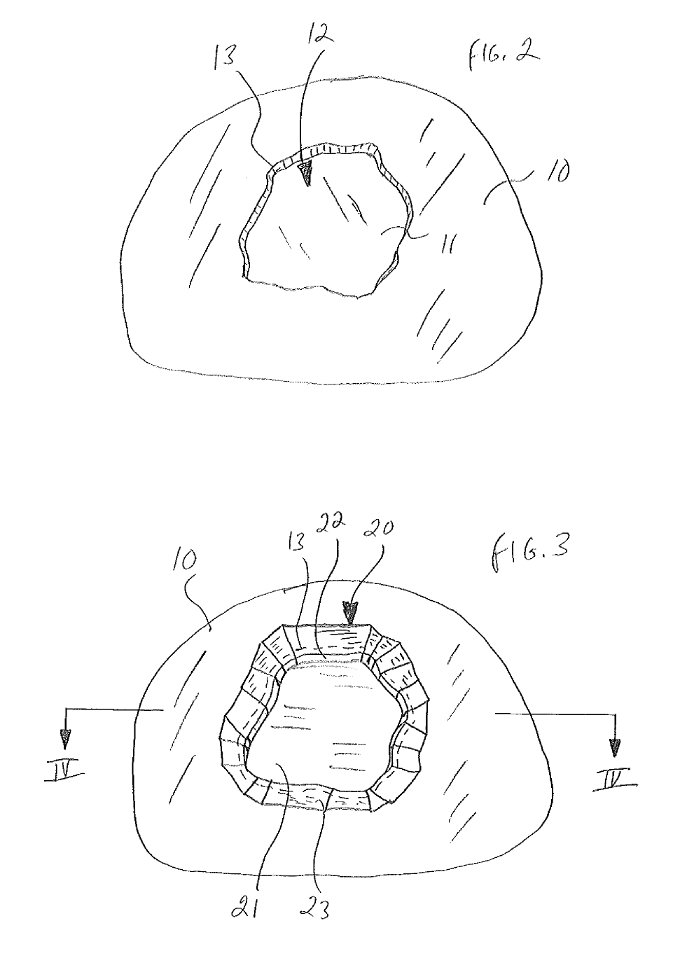

FIG. 1 illustrates an irregularly shaped defect 12 in a cranium 10, such as may have resulted from a blow to the head, the damaged bone fragments having been removed and the brain tissue 11 now being exposed. Because the edge 13 of the cranial defect 12 is of irregular shape or configuration, a custom formed replacement implant is required to best match the irregular periphery of the cranial defect 12.

A mold forming member 20 is provided to create a mold for the production of an implant 31 that substantially matches the configuration of the bone defect 12 with regard to peripheral shape, depth and curvature such that the shape of the cranium 10 after implantation of the implant 31 substantially matches the shape of the cranium 10 present prior to removal or loss of the damaged portion. As shown in the drawings, the mold forming member 20 may comprise a malleable sheet material. The thin sheet material may comprise a metal foil, a polymer film or a laminate of metal and polymer, for example. A suitable material of composition for the mold forming member 20, for example, is a thin laminate of silicone and aluminum foil. The mold forming member 20 is placed across the cavity of the defect 12 and pressed inward such that the material conforms to shape and curvature of the exposed brain tissue 11. At the edges 13 of the defect 12 the sheet material is manipulated and folded around the perimeter such that a side wall 22, outer flange or skirt 23 and mold recess 24 are created. The mold forming member preferably 20 possesses sufficient rigidity such that after manipulation within the defect 12 it will retain its molded shape after being removed from the cranial defect 12. Alternatively, the mold forming member 20 may comprise a malleable material that hardens over time or which may be cured by exposure to radiation, such as UV radiation or heat, such as for example a cross-linkable polymer film. Upon removal, the mold forming member 20 now defines a female mold 25 that substantially approximates the size, shape, depth and dimensions of the cranial defect 12.

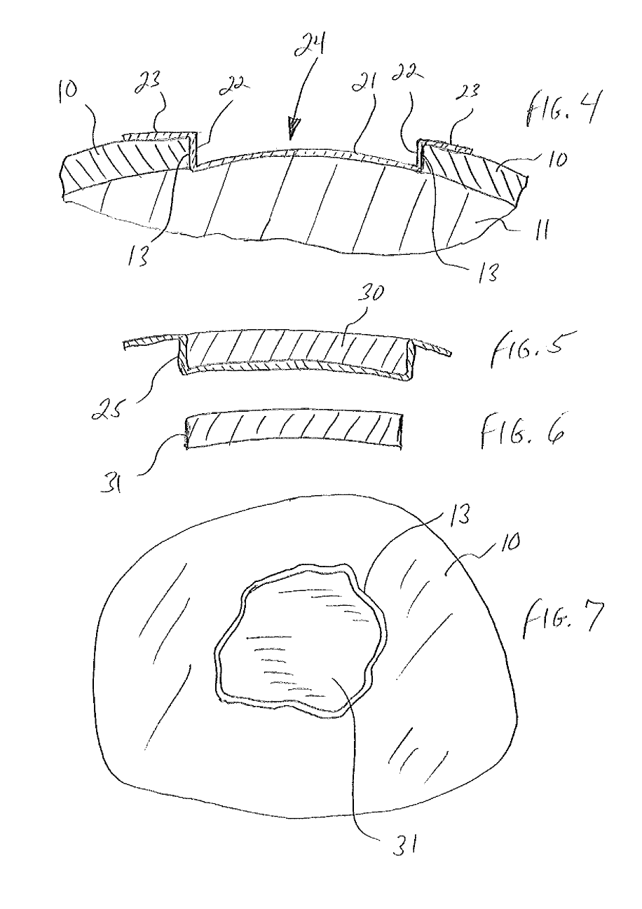

If needed a release material is poured, sprayed or brushed into the mold 25 and an implant forming material 30 is poured or otherwise introduced into the mold 25, as shown in FIG. 5. Any properly biocompatible material suitable for implantation may be used, such as a putty or cement previously approved for medical purposes, that upon curing or hardening produces a substantially rigid, structurally strong implant 31. The implant forming material 30 must have minimal shrinkage upon curing so that the dimensions of the cured implant 31 are not reduced from the dimensions of the mold 25. Most preferably the implant forming material 30 has no shrinkage, such as happens in many chemical reaction curing methodologies, or even slightly expands during curing. For example, and not meant to be limiting, a bone cement marketed under the brand name KRYPTONITE by Doctor's Research Group, Inc., has been shown to be suitable for this purpose. This bone cement comprises a first component containing a naturally occurring hydroxyl terminated fatty acid derived from castor oil, the triglyceride being synthesized with NCO groups to create pre-polymer chains ready for polymerization upon mixing with a second component also containing a naturally occurring hydroxyl terminated fatty acid derived from castor oil, plus a catalyst and water, the water reacting with the NCO groups to release carbon dioxide to produce porosity in the final material, and a third component of calcium carbonate, a non-reactive filler which enhances porosity and assists in achieving the desired mechanical and structural properties.

The exterior surface of the implant 31 may be properly shaped prior to final curing using shaping tools, such as to impart a convex surface for example. Upon curing, the implant 31 is removed from the mold 25 and further processed by cutting, shaping, abrading, polishing, etc. as needed to remove any excess material, round off edges or corners, etc. The resulting implant 31 greatly approximates the irregular shape of the defect 12, such that upon placement into the defect 12, as shown in FIG. 7, final affixation of the custom implant 25 to the cranium 10 is easily accomplished using known technology, such as through bonding, gluing, mechanical fastening or the like.

In alternative embodiments, the mold forming member 20 may comprise a putty material that can be hand formed on the bottom and sides of the defect 12 to create a thin-walled mold 25, or may a comprise a sprayable or paintable composition that is applied to the bottom and sides of the defect 12 and allowed to cure. Still further, alternative materials may be used to create the mold 25 that are cured by radiation, such as heat or UV radiation, or other means to promote cross-linking, chemical reaction, etc.

It is understood that equivalents and substitutions to certain elements set forth above may be obvious to those of ordinary skill in the art, and therefore the true scope and definition of the invention is to be as set froth in the following claims.

We claim:

1. A method of repairing a cranial defect comprising the steps of:
providing a mold forming member;
manipulating said mold forming member within a cranial defect in a cranium such that said mold folding member conforms to the shape of said cranial defect and forms a negative mold;
removing said mold from said cranial defect;
providing an implant forming material;
filling said mold with said implant forming material;
allowing said implant forming material to cure to form an implant;
removing said implant from said mold;
positioning said implant in said cranial defect; and
affixing said implant to said cranium.

2. The method of claim 1, said step of providing a mold forming member comprising providing a mold forming member composed of a malleable sheet material.

3. The method of claim 2, further comprising the step of choosing said malleable sheet material from the group of sheet materials consisting of metal foil, polymer film and laminates of polymer and metal.

4. The method of claim 1, said step of providing a mold forming member comprising providing a mold forming member composed of a putty.

5. The method of claim 1, said step of providing an implant forming material comprising providing an implant forming material composed of a biocompatible putty.

6. The method of claim 1, said step of providing an implant forming material comprising providing an implant forming material composed of a biocompatible cement.

7. The method of claim 2, said step of providing an implant forming material comprising providing an implant forming material composed of a biocompatible putty.

8. The method of claim 2, said step of providing an implant forming material comprising providing an implant forming material composed of a biocompatible cement.

9. The method of claim 1, further comprising the step of curing said mold forming member by applying radiation.

10. The method of claim 1, further comprising the step of curing said implant forming material by applying radiation.
